(19) United States
(12) Patent Application Publication
(10) Pub. No.: US 2016/0117938 A1
(43) Pub. Date: Apr. 28, 2016

(54) TASK ASSISTANCE BASED ON COGNITIVE STATE

(71) Applicant: International Business Machines Corporation, Armonk, NY (US)

(72) Inventors: James R. Kraemer, Santa Fe, NM (US); Melanie D. Polkosky, Collierville, TN (US); Puthukode G. Ramachandran, Austin, TX (US); Shunguo Yan, Austin, TX (US)

(21) Appl. No.: 14/745,615

(22) Filed: Jun. 22, 2015

Related U.S. Application Data
(63) Continuation of application No. 14/523,992, filed on Oct. 27, 2014.

(51) Int. Cl. G09B 5/00; A61B 5/145; A61B 5/02; A61B 5/12; A61B 5/00; A61B 5/01

(52) U.S. Cl. CPC G09B 5/00; A61B 5/0008; A61B 5/015; A61B 5/02; A61B 5/12; A61B 5/14

(57) ABSTRACT

Embodiments relate to providing task assistance. One aspect includes collecting sensor data from a sensor that is communicatively coupled to a computer device, and receiving inputs from a user of the device. The inputs are directed to implementation of tasks via applications operated on the device. Another aspect includes determining a cognitive state of the user from the sensor data and the inputs for each of the tasks performed and calculating a normative cognitive state of the user that is represented as a value within a range of values. A further aspect includes selecting a task assist function responsive to initiation of a task by the user at the device. The task assist function is selected based on a deviation of a value representing a current determined cognitive state from the normative cognitive state value.

### DOMESTIC PRIORITY

This application is a continuation of U.S. patent application Ser. No. 14/523,992, filed Oct. 27, 2014, the content of which is incorporated by reference herein in its entirety.

### BACKGROUND

The present disclosure relates generally to computer applications, and more specifically, to providing task assistance based on an end user's cognitive state.

A person's psychological state, or cognitive state, can have a big impact on the person's perception, acceptance, and willingness to take certain actions, and hence can impact the progression of a task undertaken by the user. A psychological state can be a good indicator of an overall experience the user has with respect to an application on a personal device, as well as with the task as a whole, and it can have a significant impact on the likelihood of future selections of applications for a task. For example, a frustrated user is less likely to select the same application or capability if he/she has other choices. In addition, a person's psychological state may change very often even during a short time, and this can affect the user's experience with an application. For example, while in a confused or doubtful state, the user may not want to continue the current task without additional help. By contrast, while in an interested or happy state, the user might be willing to accept additional activities or tasks.

### SUMMARY

Embodiments include a method, system, and computer program product for providing task assistance. The method includes collecting sensor data from at least one sensor that is communicatively coupled to a computer device, and receiving inputs from an end user of the computer device. The inputs are directed to implementation of tasks via applications operated on the computer device. The method also includes determining a cognitive state of the end user from the sensor data and the inputs for each of the tasks performed by the end user, and calculating a normative cognitive state of the end user that is represented as a value within a range of values. The values are indicative of a range of intensities of the corresponding cognitive state. The method further includes storing the sensor data, inputs, corresponding cognitive states, and normative cognitive state in a profile created for the end user, and selecting a task assist function responsive to initiation of a task by the end user at the computer device. The task assist function is selected based on a deviation of a value representing a current determined cognitive state from the normative cognitive state value.

Additional features and advantages are realized through the techniques of the present disclosure. Other embodiments and aspects of the disclosure are described in detail herein. For a better understanding of the disclosure with the advantages and the features, refer to the description and to the drawings.

### BRIEF DESCRIPTION OF THE SEVERAL VIEWS OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 depicts a cloud computing node in accordance with an embodiment;

FIG. 2 depicts a cloud computing environment in accordance with an embodiment;

FIG. 3 depicts abstraction model layers in accordance with an embodiment;

FIG. 4 depicts a block diagram of a system upon which task assistance based on cognitive states may be implemented in accordance with an embodiment;

FIG. 5 depicts a flow diagram describing a process for implementing task assistance based on cognitive states in accordance with an embodiment; and

FIG. 6 depicts an end user profile with sample data in accordance with an embodiment.

### DETAILED DESCRIPTION

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics Are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models Are as Follows:

Software as a Service (SaaS): the capability provided to the consumer is to use the provider's applications running on a cloud infrastructure. The applications are accessible from various client devices through a thin client interface such as a web browser (e.g., web-based email).The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models Are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load balancing between clouds).

A cloud computing environment is service oriented with a focus on statelessness, low coupling, modularity, and semantic interoperability. At the heart of cloud computing is an infrastructure comprising a network of interconnected nodes.

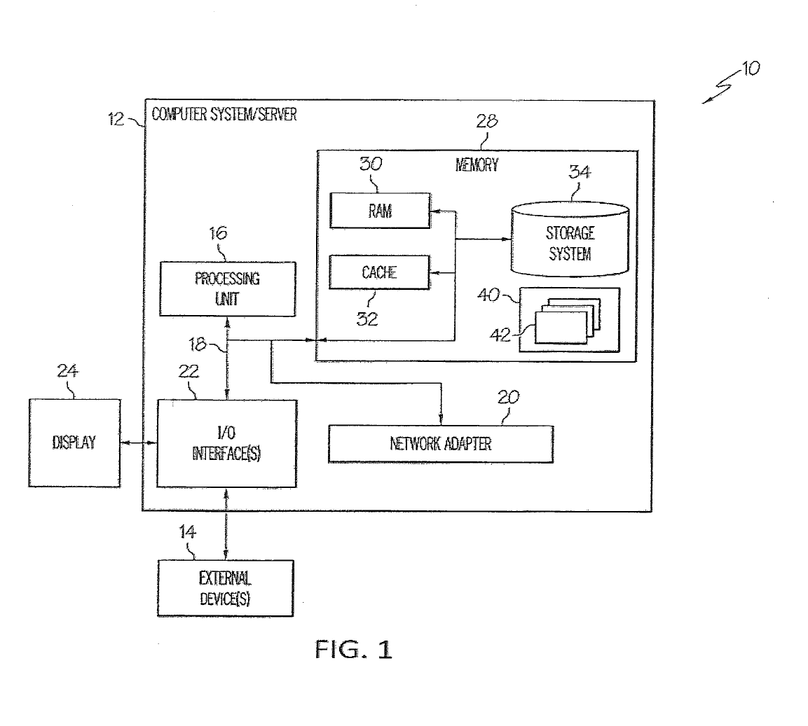

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

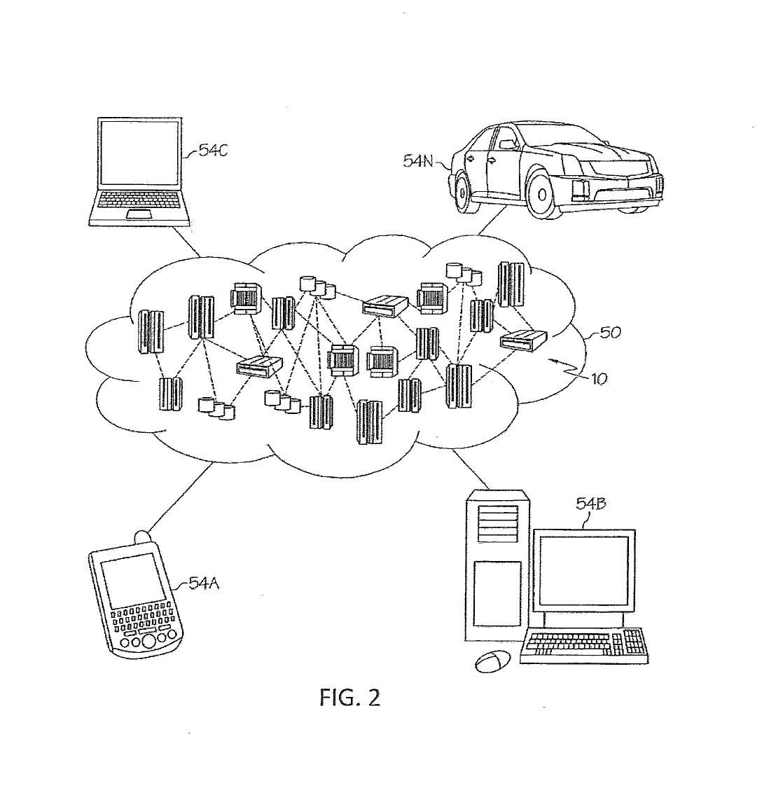

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

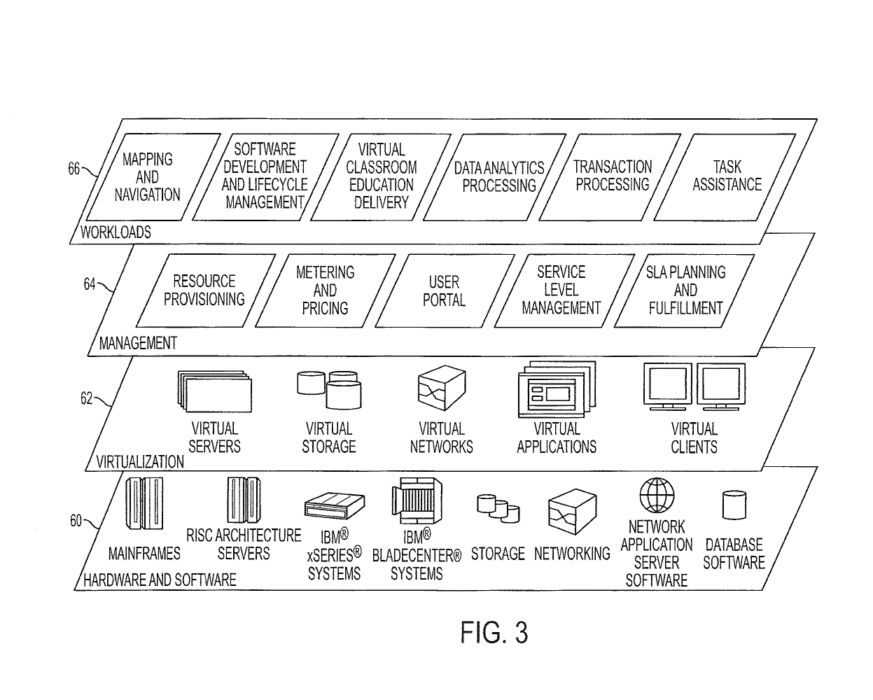

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include mainframes, in one example IBM® zSeries® systems; RISC (Reduced Instruction Set Computer) architecture based servers, in one example IBM pSeries® systems; IBM xSeries® systems; IBM BladeCenter® systems; storage devices; networks and networking components. Examples of software components include network application server software, in one example IBM WebSphere® application server software; and database software, in one example IBM DB2® database software. (IBM, zSeries, pSeries, xSeries, BladeCenter, WebSphere, and DB2 are trademarks of International Business Machines Corporation registered in many jurisdictions worldwide).

Virtualization layer 62 provides an abstraction layer from which the following examples of virtual entities may be provided: virtual servers; virtual storage; virtual networks, including virtual private networks; virtual applications and operating systems; and virtual clients.

In one example, management layer 64 may provide the functions described below. Resource provisioning provides dynamic procurement of computing resources and other resources that are utilized to perform tasks within the cloud computing environment. Metering and Pricing provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal provides access to the cloud computing environment for consumers and system administrators. Service level management provides cloud computing resource allocation and management such that required service levels are met. Service Level Agreement (SLA) planning and fulfillment provide pre-arrangement for, and procurement of, cloud computing resources for which a future requirement is anticipated in accordance with an SLA.

Workloads layer 66 provides examples of functionality for which the cloud computing environment may be utilized. Examples of workloads and functions which may be provided from this layer include: mapping and navigation; software development and lifecycle management; virtual classroom education delivery; data analytics processing; transaction processing; and task assistance.

Task assistance may be implemented through an end user's computer device based on the cognitive state of the end user. Task assistance may include modifying features and functions of an application interface and providing a help feature. For example, a modified user interface may have enlarged task features, a reduced number of task elements, and/or suppression of extraneous content, such as advertising. The cognitive state may be one of various cognitive states, such as happy, angry, anxious, stressed, frustrated, etc. When an end user is determined to have a cognitive state that is undesirable (e.g., defined as one that may negatively impact the end user's experience with the computer device), the task assistance features may be implemented to moderate the cognitive state of the end user. Likewise, when the end user is considered to have a cognitive state that is desirable, the task assistance features may include enabling or introducing advertising on the computer device. These, and other features of the task assistance based on cognitive state will now be described.

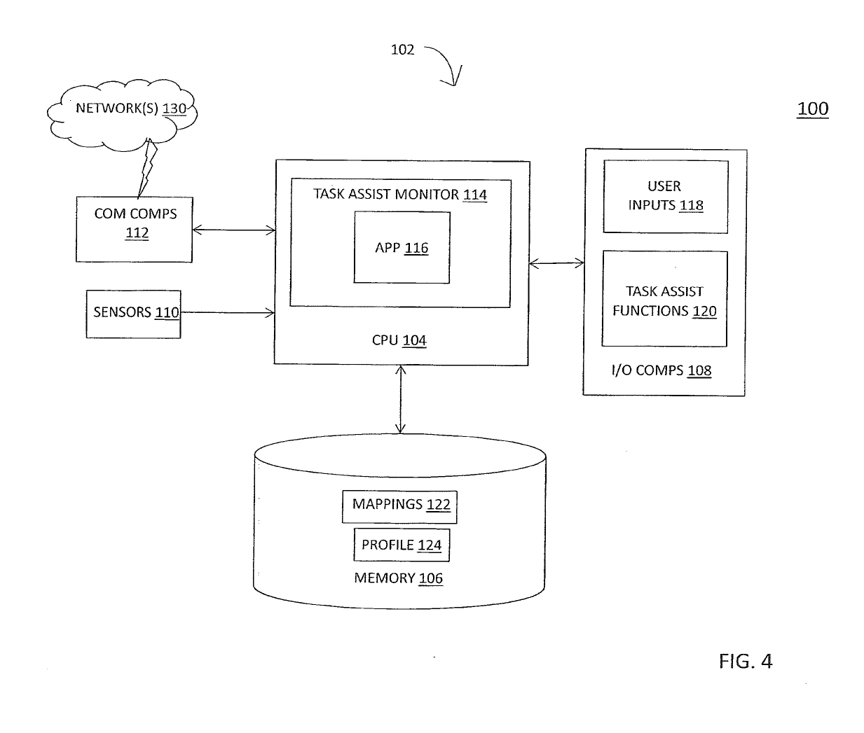

Turning now to FIG. 4, a system 100 upon which the task assistance features may be implemented will now be described in an embodiment.

The system 100 includes a computer device 102 that may be communicatively coupled to one or more networks 130 via communication components 112. The computer device 102 may be implemented as a portable electronics device (e.g., a smart phone or tablet PC). The computer device 102 includes a computer processor (CPU) 104, a memory 106, and input/output (I/O) components 108. The computer device 102, e.g., may correspond to cell phone 54A of FIG. 2, and the networks 130 may correspond to cloud computing environment 50 of FIG. 2.

The CPU 104 executes various applications (e.g., application 116) on behalf of an end user of the device 102, which applications may be stored in the memory 106. In an embodiment, the applications may alternatively reside on a server and access to the applications is provided to the computer device 102 over the networks 130 through an application interface.

The CPU 104 also executes a task assist monitor 114 for implementing the task assistance features described herein. The task assist monitor 114 may be implemented in middleware and provides monitoring of the end user's cognitive state from various inputs received, as will be described herein.

In addition to storing applications, the memory 106 may store mappings of cognitive states to associated task assist functions. The memory 106 may also store a profile of the end user that includes a compilation of cognitive states of the end user determined over time, as well as associated task assist functions that were determined to facilitate implementation of specified tasks for the end user. The success of the task performance may be determined as a function of the end result of the task (e.g., completed or not completed, time span of task execution, repeated inputs, etc.), as well as a moderation of the cognitive state of the end user as compared to the cognitive state at the beginning of the task.

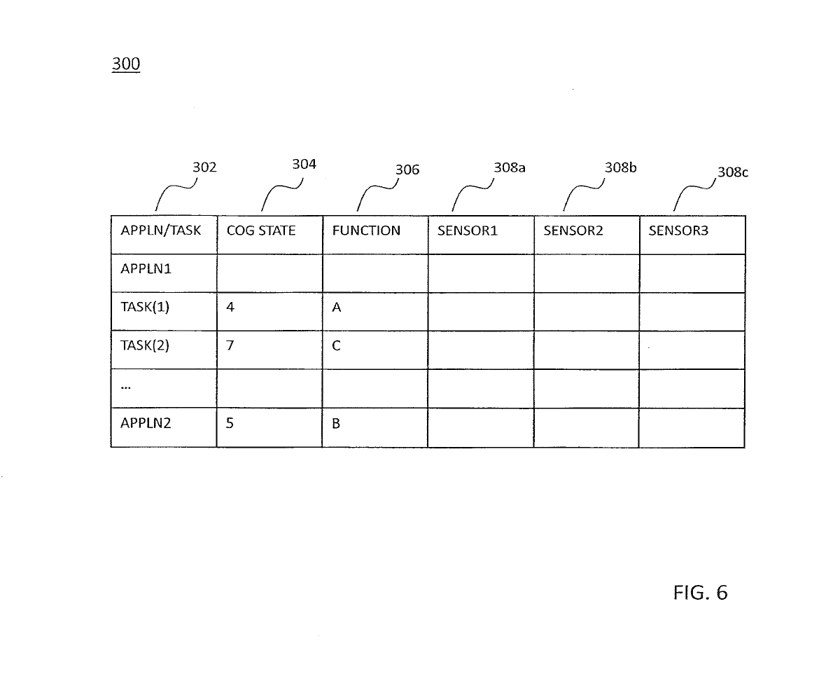

The profile may store a normative cognitive state of the end user calculated, e.g., as an average of previously determined cognitive states under similar circumstances (e.g., where the end user performs the same task or similar task, or the sensor data indicates similar conditions exist as compared to previously detected conditions). A profile is further described in FIG. 6.

The I/O components 108 may include one or more of a microphone, key board, keypad, touch screen, display screen, and speakers. End user inputs 118 to perform one or more tasks are received by the computer device 102 from at least one input element of the I/O components 108. In addition, one or more output elements of the I/O components 108 present an application interface for enabling the end user to enter inputs for performing a task, and the task assist monitor 114 can modify the interface based on certain criteria and present the modifications via the output elements (as task assist functions 120).

The computer device 102 further includes one or more sensors 110 that are communicatively coupled to the computer device 102 and provide sensor data to the task assist monitor 114. The sensors 110 may include biometric sensors (e.g., sensors that measure an individual's oxygen levels, blood sugar, heart rate, pulse, respiration, etc.), thermal sensors that measure ambient temperatures, voice analysis sensors and logic that analyze voice inputs through the microphone (e.g., one of I/O components 108) to determine a stress level or other voice quality, a camera or image sensor and logic that captures facial expressions and assesses the expressions to determine a cognitive state, and an accelerometer to measure speed and motion, to name a few. Other possible metrics subject to data collection include vibration, lighting conditions, geo-locations, and the presence of environmental chemicals.

Sensor data can be useful in assessing the current cognitive state of the end user. For example, an accelerated heart rate, heightened voice or pitch, etc., can be an indicator of stress, anxiousness, or anger. The task assist monitor 114 uses the sensor data to not only determine a cognitive state of the end user, but also to assess a degree of intensity of the cognitive state. For example, a level of stress can be evaluated as moderate or severe based on the particular values of the sensor data (e.g., a pulse of 100 may be attributed to moderate stress, while a pulse of 120 may be attributed to severe stress). The sensor data can be used in combination (e.g., multiple sensor data inputs) to evaluate the end user's cognitive state. For example, a data value signifying fast movement of the end user (via the accelerometer) may be the actual cause of a higher-than-normal pulse, rather than stress. Alternatively, a high-than-normal pulse, coupled with a facial expression captured by the image sensor may point to a cognitive state of stress.

In an embodiment, the sensor data is analyzed in conjunction with tasks initiated or in process by the end user in determining a cognitive state. In this embodiment, indicators such as repeated selection of a key or set of key strokes, an unusually long duration of time to execute the task, or a duration of time in which input is provided but is anticipated, may point to a cognitive state of stress.

In a further embodiment, the task assist monitor 114 can access and review system and/or application event logs to determine conditions that may indicate an undesired cognitive state. For example, an event log that shows multiple network outages or drops within a relatively short period of time can contribute to a heightened level of stress.

In a further embodiment, the task assist monitor 114 can utilize geo-location information in determining a cognitive state. For example, if the end user's locations are monitored over time and a pattern of undesirable cognitive states are determined when the user is at or near a particular location, the task assist monitor 114 can infer that the end user has or will have that cognitive state. The geo-location information may be derived from satellite or cellular positioning data via the communication components 112 and networks 130.

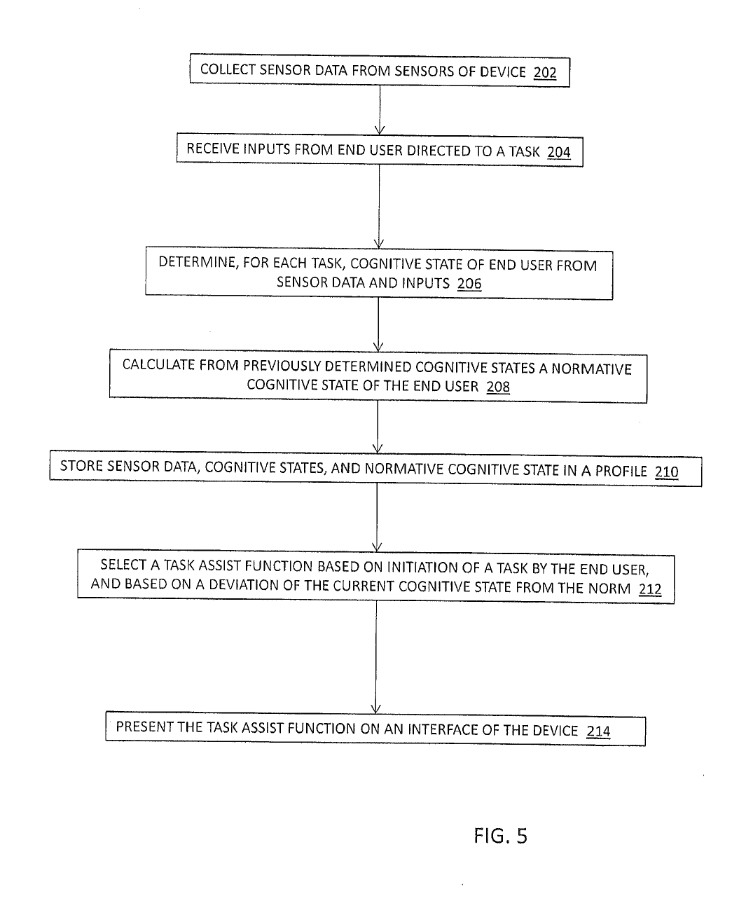

Turning now to FIG. 5, a flow diagram describing a process for implementing the task assistance features will now be described.

At block 202, the task assist monitor 114 collects sensor data from at least one of the sensors 110 coupled to the computer device 102.

At block 204, the task assist monitor 114 receives inputs from the end user of the computer device 102. The inputs are associated with one or more tasks the end user has initiated through an application. For example, the tasks may be directed to a banking operation, such as transferring funds between accounts or making a payment.

At block 206, the task assist monitor 114 determines a cognitive state of the end user from the sensor data and the inputs received for the tasks or in pursuit of implementing the tasks.

At block 208, the task assist monitor 114 calculates, from previously determined cognitive states over a period of time for the same or similar tasks, a normative cognitive state of the end user. The normative cognitive state may be represented as a value within a range of values that indicate a range of intensity of the corresponding cognitive state. For example, for a cognitive state, ‘stress,’ a range may be from 0 to 10, where 0 reflects no stress and a 10 reflects a highest level of stress.

At block 210, the task assist monitor 114 stores the sensor data, inputs, corresponding cognitive states, and normative cognitive state in a profile created for the end user. It will be understood that this information may be stored as it is received, collected, and/or calculated. As shown in FIG. 6, a profile 300 may include an identification of applications and tasks (column 302), a normative cognitive state for the applications/tasks (column 304), a task assist function (column 306), and sensor data from sensors (columns 308a, 308b, 308c). The task assist function value in column 306 may identify the task assist function determined to be most successful in moderating the cognitive state of the end user based on past activities. This value may change or become updated based on future results of its application.

Turning back to FIG. 5, once the profile is created, the task assist monitor 114 can monitor further sensor data as well as future inputs, and provide a task assist function that is customized to the end user's cognitive state and sensitivities. As described in block 212, the task assist monitor 114 selects a task assist function responsive to initiation of a task by the end user at the computer device 102. The task assist function may be selected based on a deviation of a value representing a current cognitive state from the normative cognitive state value.

At block 214, the task assist function is provided to the end user via a modified interface on the computer device.

In an alternative embodiment, if the cognitive state is determined to be within a desirable range (e.g., very low stress), the task assist function may include providing supplemental information, e.g., advertising, to the end user.

In a further embodiment, the task assist monitor 114 analyzes the profile over time to identify any patterns among the determined cognitive states, the user inputs, and the sensor data. Once a pattern has been identified, the task assist monitor 114 may predict a cognitive state of the end user before the end user performs a task when current sensor data and contextual user activity suggest that a particular cognitive state is anticipated. The task assist monitor 114 can select a task assist function based on this predicted cognitive state.

In a further embodiment, the task assist monitor 114 can be initialized using cognitive states and mappings to task assist functions that have been determined to facilitate execution of the tasks for a generalized population of end users. With respect to the end user who has no history (e.g., profile), the mappings may be used initially for the end user in selecting task assist functions.

Technical effects of the embodiments include facilitating task assistance through an end user's computer device based on the cognitive state of the end user. Task assistance may include modifying features and functions of an application interface and providing a help feature. For example, a modified user interface may have enlarged task features, a reduced number of task elements, and/or suppression of extraneous content, such as advertising.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), astatic random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for providing task assistance, the method comprising:
collecting, from a computer device, sensor data from at least one sensor that is communicatively coupled to the computer device;
receiving inputs from an end user of the computer device, the inputs directed to implementation of tasks via applications operated on the computer device;
determining, by the computer device, a cognitive state of the end user from the sensor data and the inputs for each of the tasks performed by the end user;
calculating a normative cognitive state of the end user that is represented as a value within a range of values, the values indicative of a range of intensities of the corresponding cognitive state;
storing the sensor data, inputs, corresponding cognitive states, and normative cognitive state in a profile created for the end user; and
selecting, by the computer device, a task assist function responsive to initiation of a task by the end user at the computer device, the task assist function selected based on a deviation of a value representing a current determined cognitive state from the normative cognitive state value.

2. The method of claim 1, further comprising:
identifying, from the profile, patterns among the determined cognitive states, the inputs, and the sensor data;
predicting a cognitive state of the end user prior to implementation of a task based on the patterns in view of currently acquired sensor data and contextual end user activity; and
selecting a task assist function based on the predicted cognitive state.

3. The method of claim 1, wherein the sensor data is derived from sensors, the sensor data including at least one of:
ambient temperature;
biometric data comprising at least one of:
oxygen;
blood sugar;
heart rate; and
respiration rate;
vibration;
acceleration;
lighting conditions;
environmental chemical presence;
noise levels;
voice;
images; and
geo-locations.

4. The method of claim 1, further comprising:
mapping cognitive states to task assist functions and tasks, the task assist functions determined to facilitate execution of the tasks for a generalized population of end users.

5. The method of claim 1, wherein the task assist function comprises a modified user interface having enlarged task features.

6. The method of claim 1, wherein the task assist function comprises a modified user interface having reduced number of task elements.

7. The method of claim 1, wherein the task assist function comprises a help feature.
